(19) United States
(12) Patent Application Publication
(10) Pub. No.: US 2025/0241581 A1
(43) Pub. Date: Jul. 31, 2025

(54) System and Method for the Non-Invasive Detection of Spreading Depolarization using EEG

(71) Applicant: CARNEGIE MELLON UNIVERSITY, Pittsburgh, PA (US)

(72) Inventors: Alireza CHAMANZAR, Pittsburgh, PA (US); Pulkit GROVER, Pittsburgh, PA (US)

(73) Assignee: CARNEGIE MELLON UNIVERSITY, Pittsburgh, PA (US)

(21) Appl. No.: 18/689,207

(22) PCT Filed: Sep. 29, 2023
(86) PCT No.: PCT/US23/34099

§ 371 Date: Mar. 5, 2024

Related U.S. Application Data
(60) Provisional application No. 63/413,809, filed on Oct. 6, 2022.

(51) Int. Cl. A61B 5/374; A61B 5/00; G16H 30/40

(52) U.S. Cl. CPC A61B 5/374; A61B 5/7267; G16H 30/40

(57) ABSTRACT

Disclosed herein is a system and method implementing a processing and detection pipeline to detect spreading depolarization waves in a brain occurring after a traumatic brain injury. The method relies solely on EEG data collected by a standard EEG machine.

### RELATED APPLICATIONS

This application is a national phase filing under 35 U.S.C. § 371 claiming the benefit of and priority to International Patent Application No. PCT/US23/34099, filed Sep. 29, 2023, entitled “System and Method for the Non-Invasive Detection of Spreading Depolarization using EEG”, which claims the benefit of U.S. Provisional Patent Application No. 63/413,809, filed Oct. 6, 2022, the contents of which are incorporated herein in their entirety.

### GOVERNMENT INTEREST

This invention was made with U.S. government support under contract 1763561 awarded by the National Science Foundation. The U.S. government has certain rights in the invention.

### BACKGROUND

Background spreading depolarizations (SDs) are waves of neurochemical changes in the brain, which propagate slowly (1-8 mm/min) across the cortical surface and suppress neural activity. SDs are caused by a breakdown in the ionic homeostasis across neuronal membranes after traumatic brain injury (TBI). Increasing evidence shows that SDs are associated with poor clinical outcomes in traumatic brain injuries, strokes, and hemorrhages and that this association is causal, such that the neurophysiological sequelae of SDs directly worsen secondary brain injury. SDs, if detected, can be used as a biomarker to predict worsening brain injuries.

The Non-Invasive detection of SDs could transform critical care for brain injury patients but has remained elusive. Current methods to detect SDs require surgeries to acquire invasive intracranial recordings, which have limited spatial coverage. Such surgeries are risky and unlikely to be recommended to patients with milder brain injuries.

Each year, around 69 million people worldwide suffer from TBI with 2.5 million patients in the United States. Recent data on five-year outcomes for patients with moderate and severe TBI in the United States indicate that more than half of these patients experienced neurological worsening or death. Therefore, detection of SD as a reliable biomarker and potential therapeutic target may help improve clinical outcomes.

### SUMMARY

Disclosed herein is a system and method for the automated, non-invasive detection SDs detection using scalp electroencephalography (EEG) for patients with severe TBI. The method has learnable parameters and improved velocity estimation and is able to extract and track propagating power depressions using low-density EEG.

The method provides the advantages of detecting SDs without the need for exposing the patient to risky, intracranial surgery. Additionally, the method is likely to be used on a wider range of patients (i.e., patients with milder TBIs) who would not normally be recommended for surgery, thus providing a way to detect SDs in a wider group of patients. Lastly, the method is less expensive to use than prior art methods requiring surgical intervention.

### BRIEF DESCRIPTION OF THE DRAWINGS

By way of example, a specific exemplary embodiment of the disclosed system and method will now be described, with reference to the accompanying drawings, in which:

FIG. 1a shows the position of 11 ipsilateral EEG electrodes and 8 contralateral electrodes. FIG. 1b is a graph showing 3 hours of the EEG recording across ipsilateral and contralateral electrodes.

FIGS. 2(a,b) are graphs showing the effect and results of preprocessing steps on actual data.

FIG. 3 is a block diagram showing the steps of the disclosed method.

FIG. 4 shows the ipsilateral scalp electrode locations in 2D images.

FIG. 5 is a diagram showing the mapping of optical flows on the scalp spherical surface.

FIGS. 6(a,b) show a visualization of effective propagation measures (EPM).

FIGS. 7(a,b) show a visualization of a single isolated SD event.

FIGS. 8(a-c) show an evaluation of the performance of the disclose method using a cross-validation analysis.

FIG. 9 is a histogram of the estimated speed of propagation for detected SD waves.

FIG. 10 is a flowchart showing the steps of the method.

### DETAILED DESCRIPTION

The disclosed invention for non-invasive SD detection in severe TBI patients uses an automated algorithm applied to real data collected using standard scalp EEG (a low-density EEG system with 19 electrodes at 10-20 standard locations). The system was verified by comparing results from a group of severe TBI patients who underwent decompressive hemicraniectomy (DHC) surgery, followed by continuous monitoring in an intensive care unit (ICU) using simultaneous scalp EEG and intracranial electrocorticography (EcoG) recordings.

Dataset The dataset was obtained as part of a multicenter observational clinical study that monitored SDs in TBI patients. Continuous EEG signals were recorded over a period of several days (95±42.2-hour on average) following DHC using a DC-coupled EEG amplifier with a sampling frequency of 256 Hz, from 19 electrodes placed at 10-20 standard locations.

In addition, during the DHC procedure, a strip of six monopolar ECoG electrodes (all the ECoG and EEG electrodes were referenced to a common electrode on scalp), with an interelectrode distance of 1 cm, was placed on the hemisphere that underwent the DHC, and continuous ECoG and EEG data were recorded simultaneously using the same amplifier. The recorded ECoG signals were visually assessed by a clinical expert to identify and annotate the SD episodes in the dataset.

For EEG preprocessing and artifact removal two additional electrophysiological recordings were used: (i) an electrocardiogram (ECG) signal recorded at a 500 Hz sampling frequency and (ii) a plethysmogram (PLETH) signal at a 125 Hz sampling frequency, recorded using a bedside patient monitor.

Data from 12 (nine male and three female) patients with severe TBI were collected. Two patients had DHC in the left hemisphere, and the remaining 10 patients had DHC in the right hemisphere. Eleven patients experienced subdural hematoma (SDH), and one patient had an epidural hematoma (EDH).

Each SD event in these TBI patients was annotated over time by a clinical neuroscientist through visual assessment of full-band ECoG signals. In this paper, an SD event refers to a unique SD wave, as annotated by consideration of all electrodes of the ECoG strip. Each unique SD wave was annotated at the start of a slow near-DC negative shift (in 1-10 MHz), i.e., slow potential change (SPC), in a chosen ECoG electrode, which is no t always the same, even for the same patient. It should be noted that the temporal annotation of an SD event through visual inspection of the EcoG signals may not accurately reflect the actual onset of each SD wave.

Four different types of SD events were annotated: (i) CSD: an event during which there was a cortical spreading depression (CSD) in each electrode that had an SPC, where CSD was a manifestation of spreading depolarization and was defined as a cortical wave of depression in the high-frequency (>0.5 Hz) ECoG (HF-ECoG) signals40; (ii) ISD: an event where the HFECoG signals at all the participating electrodes with SPC were already flat—these are called isoelectric spreading depolarizations (ISDs); (iii) CSD/ISD: an event where some ECoG electrodes experienced CSD-like propagation, while other electrodes had ISDs; and (iv) scCSD: an event which was identified as a clear SD in the signal of a single electrode. Although it appeared only on a single electrode, it met the consensus criteria, defined by Co-Operative Studies on Brain Injury Depolarizations (COSBID), to be classified as an SD. According to COSBID, a minimal criterion to score an event as SD is “an event which has a characteristic DC shift associated with spreading depression of spontaneous activity even if DC shift and spreading depression are restricted to a single channel.”

Ground truth SDs were annotated based on a single ECoG strip located in the DHC hemisphere, with no invasive measurements in the contralateral hemisphere (i.e., the hemisphere with an intact skull). The scalp EEG signals from the contralateral hemisphere and ipsilateral hemispheres were significantly different due to the missing part of the skull in the ipsilateral hemisphere (e.g., ipsilateral EEG signals had higher average power. The enhancement of EEG signals due to the defects in the skull is consistent with observations of “breach rhythm” reported in the literature, which is an increase in signal power in a wide range of frequencies in areas with skull defects.

For testing purposes, only ipsilateral scalp EEG electrodes for each patient were used because of the missing spatial SD ground truth in the contralateral hemisphere, and heterogeneity of the baseline EEG power between the hemispheres with DHC and the hemisphere with an intact skull. In practice, EEG data will be collected from hemispheres having an intact skull.

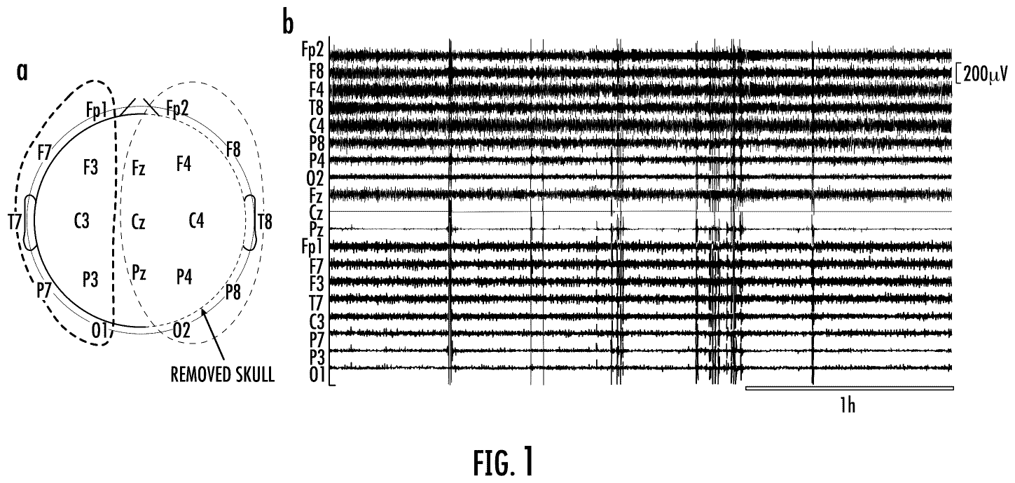

For all of the 12 patients in the dataset, there is a statistical difference (p<1×e⁻⁸) between the ipsilateral and contralateral average power. However, even in the hemisphere with DHC, the scalp electrodes which are far from the site of surgery have overall lower baseline power, in comparison to the electrodes which are placed right on top of the regions with a missing skull. FIG. 1 shows the EEG baseline power in a patient with right DHC. The EEG baseline power in ipsilateral (with missing skull) and contralateral (with intact skull) hemispheres in a patient with right decompressive hemicraniectomy (DHC). FIG. 1a shows 11 ipsilateral EEG electrodes marked with red dashed line, and 8 contralateral electrodes marked with blue dashed line. FIG. 1b shows 3 hours of the EEG recording across ipsilateral (red trace) and contralateral (blue trace) electrodes. Most of the ipsilateral electrodes on top of the DHC region (with missing skull) had higher EEG baseline power (e.g., Fp2, F8, F4, T8, C4, and P8), in comparison with the contralateral electrodes on the regions with intact skull. The EEG signals were preprocessed as described below (bandpass filtered in [0.5, 30] Hz). The signal at Cz had poor quality and was removed through the preprocessing steps.

Preprocessing Pipeline The collected EEG data is first preprocessed using a preprocessing pipeline to prune the continuous EEG recordings and reject ICU-related artifacts and segments of EEG signals with poor quality electrode-scalp contacts.

The EEG data is first band-pass filtered and downsampled. In one embodiment, MATLAB's EEGLAB toolbox43 can be used for filtering and downsampling. To evaluate the performance of the disclosed method in different frequency bands, the EEG signals were bandpass filtered in different frequency ranges, namely, [0.001, 0.01] Hz (near-DC), [0.5, 4] Hz (Delta), [4, 8] Hz (Theta), [8, 12] Hz (Alpha), and [12, 30] Hz (Beta) using a Hamming-windowed sinc finite impulse response (FIR) filter. The filter order was 1000 with a transition bandwidth of ^{˜}0.02 Hz. An upper cutoff frequency of 30 Hz was used to remove high-frequency noise components. The filtered EEG signals were then downsampled to 64 Hz. The ECG and PLETH signals were also bandpass filtered in the frequency range of [0.5, 30] Hz and then downsampled to 64 Hz.

There may be segments of the EEG recordings that are of poor quality due to less than optimal electrode-scalp contacts. This could occur, for example, as the result of movements of patient on the bed and conductive gel/saline drying out at each electrode, etc. CNS Advanced ICU EEG Amplifier monitors the quality of each electrode-scalp contact through continuous impedance recording at a sampling frequency of 1 Hz. To enable the disclosed method to detect SDs even when a few of the electrodes do not have good contact, impedance recordings were used to implement masks for automated removal of the parts of EEG signals with poor quality EEG. The continuous recording of impedance at each EEG electrode are upsampled to match the sampling frequency of the EEG signals. For each electrode, the median of impedance over the whole recording is calculated (M_{Imp}^{ch}) and parts of the EEG signals with abnormally high (>2 M_{Imp}^{ch}) impedance were used for masking. Instead of cutting these parts out of the EEG signals, these parts are assigned dummy zero values (i.e., to mask out these parts) to maintain the continuity of the recordings. These masked-out sections were excluded from the power envelope extraction and the downstream analyses (described later) to prevent false alarms resulting from these zero values. This allows keeping the parts of the dataset where a few of the electrodes have good recording quality.

For the time intervals during which the signals of all EEG electrodes were masked out, dummy zero values are assigned to the PLETH and ECG signals as well for performing independent component analysis, discussed next.

Next, artifacts were classified and removed using independent component analysis (ICA). The ECG, PLETH, and EEG signals are grouped together and n ICA is performed to extract and remove sources of artifact (such as eye blinks, eye movements, heartbeats, and muscle artifacts) in EEG signals. In one embodiment, the EEGLAB43 toolbox is used to calculate the independent components and an automated EEG independent component classifier plugin (ICLabel) in EEGLAB is used to guide the decision on which components belong to sources of artifacts and those components are subsequently removed from the EEG recordings.

Extracted ICA components may not perfectly separate some artifacts with abnormally high amplitudes (i.e., outliers) from the EEG signals. Therefore, as the last step in the preprocessing pipeline, the amplitude outliers are detected and removed. In one embodiment, the Tukey's fences method is used to remove the outliers. Tukey defines the outliers as data points that fall outside an interquartile range of [Q₁−k(Q₃−Q₁), Q₃+k(Q₃−Q₁)], where Q₁ and Q₃ are the first and third quartiles respectively, and k is an outlier threshold. In one embodiment, k=3 is used, which detects far out data points according to Tukey's outlier definition. For each electrode parts of the EEG signals with outlier amplitude, were detected and masked out (following the same procedure as impedance-based masking discussed earlier).

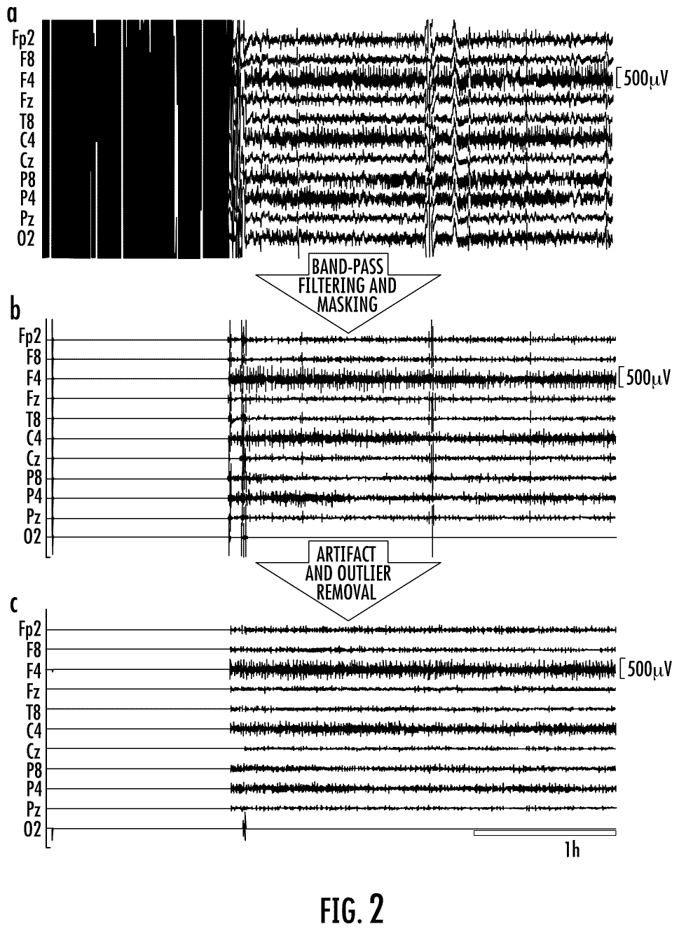

FIG. 2 illustrates the preprocessing steps applied on exemplary data comprising the ipsilateral EEG signals of a patient with right DHC, in a 4-hour time window. FIG. 2a shows the full-band EEG signals, with poor quality (high impedance) in the first ^{˜}77 min of the recording, which is masked out in the band-pass filtered signals (in Delta band, see FIG. 2b). FIG. 2c shows the detection and removal of artifacts and outliers. The preprocessed continuous EEG signals were then used for SD detection using the disclosed method.

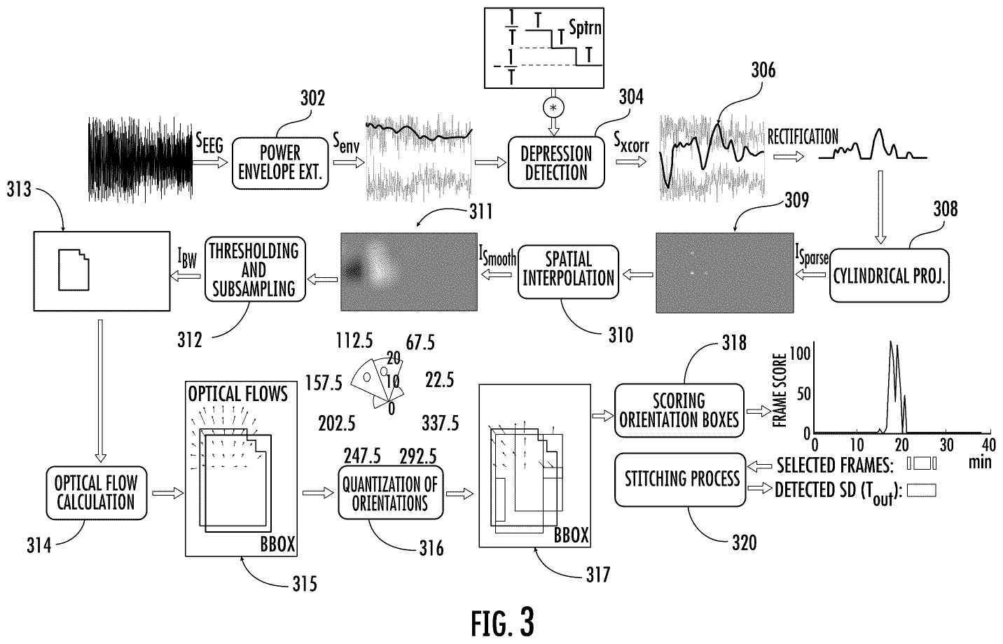

Detection Pipeline The steps of the main detection method are shown in FIG. 3. The method is an automated SD detection framework with intuitive steps and interpretable detection outputs and results. It addresses the challenges of noninvasive detection of SD waves in EEG by breaking down the challenging task of detecting the whole propagating SD wave in the brain using noisy and blurry filtered scalp EEG signals into simpler tasks of detection and classification of disjoint SD wavefronts.

The steps of the method are outlines here and described in more detail later herein. First, at 202, the power envelope (Sₑₙᵥ) of the scalp EEG signals (S_{EEG}) at each electrode are extracted. At steps 204, the power envelope is cross-correlated with a first-derivative kernel (S_{Ptrn}) to detect depressions (power reductions). The detected power depressions are expressed as large positive peaks in S_{corr}, as shown in 206. These detected depressions are then projected onto a 2D plane using cylindrical projection at 208 to obtain depression wavefronts. The resulting image (I_{Sparse}) is then spatially interpolated at 210, thresholded and subsampled at 212 to obtain binary images (I_{BW}), where the depression wavefronts were captured as white contiguous pixels. Over time, a time series of binary images is formed. Propagating SD wavefronts are detected and tracked based on their speed and direction of propagation. To estimate the speed and direction of propagation of depression wavefronts on these 2D planes, a computer vision technique called optical flow is used at 214. Dominant directions of propagation (marked bins in the orientation histogram) were found through quantization of orientations at 216 and scored at 218 based on the consistency and speed of propagation of wavefronts. Candidate frames were selected based on the calculated scores and stitched together at 220 for the final detection output (Tₒᵤₜ). This method overcomes the challenge related to the effects of sulci and gyri and enables the detection and tracking of complex patterns of propagation.

Envelope Extraction—At step 302, epochs are extracted from the preprocessed EEG signals (S_{EEG}) using overlapping time windows. In one embodiment, the overlapping time windows are 240 min in length with a step size of 180 min. For each epoch, the EEG signal at each electrode (S_{EEG}) was normalized by its estimated standard deviation.

This epoching and power normalization step addresses the heterogeneity of the baseline EEG power across electrodes and over time and helps to detect and extract the depressions in electrodes with low baseline power, which are otherwise missed in the interpolation and thresholding step. In addition to the baseline power normalization, this epoching helps in the parallelization of the downstream data analysis and the training process explained later.

Following the normalization step, the amplitude values were squared, and upper root-mean-square (RMS) envelopes of the power signals were extracted using a sliding time window of 5 min. In some instances, there might be some small and isolated valid portions of the EEG signals at each electrode with the normal recording quality, which were interleaved by dummy zero values following the masking out step described above. These portions were not large enough to capture the slow depressions of SDs across EEG electrodes. To prevent false alarms resulting from these isolated intervals, at each epoch and before power envelope (Sₑₙᵥ) extraction, small intervals of the EEG signals which were less than 20 min long, and isolated, (i.e., more than 1 min apart from the nearest valid intervals) were masked out. The masked-out sections of the EEG signals were excluded from the envelope extraction.

Power Depression Extraction—In step 304, the power depressions at each electrode were detected and extracted based on the power envelopes (Sₑₙᵥ). To detect the falling edges of the power envelopes, which are followed by a prolonged power reduction, Sₑₙᵥ was cross-correlated with a piecewise-constant function as a first-order derivative kernel (see Sₚₜᵣₙ kernel in FIG. 3). This kernel extracts EEG power depressions as large positive values in the cross-correlated signal (S_{xcorr}). The 5 min width used for envelope extraction and depression edge detection is small in comparison to the large temporal width of depressions in severe TBIs. The same first-order derivative kernel was used for the detection of DC shifts in the near-DC components ([0.001, 0.01] Hz). This kernel was directly applied on S_{EEG} after the epoching and power normalization step, to detect the falling edges of SPCs in the near-DC components.

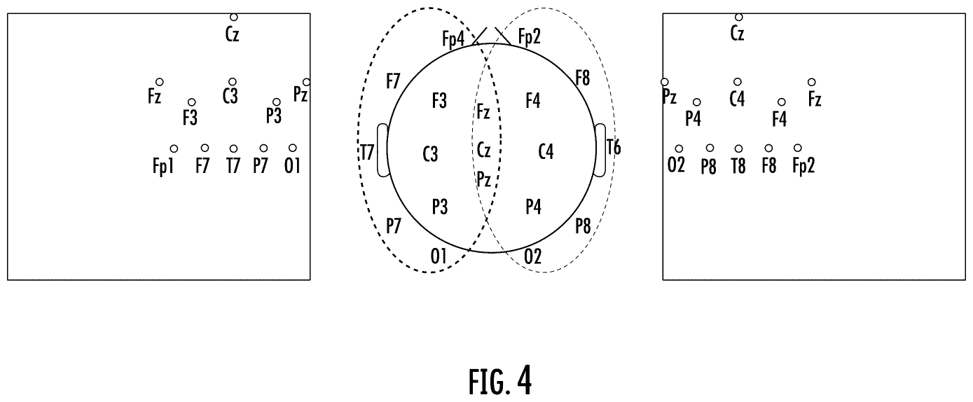

Cylindrical Projection—At step 308, the S_{xcorr} signals extracted from ipsilateral EEG electrodes were projected at locations on a 2D plane. Before this projection, S_{xcorr} signals are rectified (i.e., negative values corresponding to the rising edges in the power envelopes were zeroed, and positive values, which correspond to the falling edges of the depressions, were kept unchanged). FIG. 3 shows an example of the resulting image 309 (I_{Sparse}) using this projection, around an annotated SD event. The corresponding scalp electrode locations in these 2D plane are shown in FIG. 4 for patients with left and right DHC. At each time point, the median value of the S_{xcorr} signals at ipsilateral electrode locations was assigned to the rest of the pixels in I_{SParse}.

Interpolation and Thresholding—At step 310 I_{SParse} is spatially interpolated using a 2D Gaussian kernel with σ=2.62 cm. This large standard deviation was chosen because of the low density of EEG electrodes, where the average inter-electrode distance is ˜5.4 cm. For interpolation at the boundaries, each I_{SParse} image was padded with the median of the corresponding ipsilateral values of the S_{xcorr} signals. The resulting smooth image (I_{Smooth}) is shown in FIG. 3 as 311. At step 312, a thresholding mechanism is introduced to extract binary images (I_{BW}) from the smooth 2D images (I_{Smooth}): (i) zeros are assigned zeros to the pixels at each image whose values were ≤M_{I}_{_{Smooth}}+Thr₁(H_{I}_{ₛₘₒₒₜₕ}−M_{I}_{ₛₘₒₒₜₕ}), where M_{I}_{ₛₘₒₒₜₕ} and H_{I}_{ₛₘₒₒₜₕ} are the median and the maximum of the pixel values at each I_{Smooth} image respectively, and Thr₁ is the depression-level threshold at each time point which were found through the training process as a learnable parameter

This step zeros the pixels with values close to or less than the median value at each image, and sets the pixels with large positive values in I_{Smooth} as candidates for SD wavefronts; (ii) In addition, zeros are assigned to the whole images where most of the EEG electrodes were masked out (i.e., images with less than 5 participating scalp electrodes out of the total 11 ipsilateral electrodes). This rejects the binary images with poor EEG signals; (iii) the remaining pixels were set (i.e., assign a 1 value) and finally rejected pixels (i.e., assigned a 0 value) the images where more than half of the pixels are set. This was done with the reasonable assumption that SD depressions cannot spatially expand over more than half of the cortical surface. Following this three-stage thresholding mechanism, binary images (I_{BW}, 313, in FIG. 3) were extracted, where non-zero pixels form connected components which are either parts of the SD wavefronts or non-SD activities in the brain. Through the following steps, these connected components were classified and detected and SD wavefronts were tracked.

Subsampling and Optical Flow Calculation—At step 312, the time series of binary images 313 were temporally subsampled, in one embodiment, every 30 s. Because the extracted power envelopes and depression signals (S_{xcorr}) have a very slow temporal pattern (in the order of minutes), this temporal subsampling significantly reduces the computational complexity of the method without adversely affecting the SD detection performance.

In addition, due to the very low density of the CNS EEG grid (only 19 electrodes), the I_{BW} images were spatially sampled so that the inter-electrode distances in these 2D images are less than three pixels. This helps to better capture the propagation of SD wavefronts across EEG electrodes in these binary images and further reduces the computational power required for the downstream analysis. The bicubic interpolation method was used for spatial subsampling of I_{BW}—The parameters of the interpolation kernel (standard deviation a) and the spatial subsampling rate were carefully chosen so that the corresponding pixels of each electrode location on the scalp have a representation in the lower resolution binary image. Therefore, it is not expected that any connected component (wavefront) in the lower resolution binary image would be missed. After the subsampling steps, at step 314 closely followed the steps in ref. 30 for the optical flows are calculated to capture the movements of connected components and SD wavefronts.

An optical flow is a computer vision technique to track moving objects across frames of a video. It uses the spatiotemporal brightness variations of the pixels to estimate the velocity (magnitude and direction) of the moving objects. The optical flows of the depressions are calculated based on the 2D images, which are the cylindrical projections of scalp electrode locations onto a 2D plane. In these 2D images, the horizontal dimension captures the azimuthal angle (ϕ), and the vertical dimension captures the polar angle (θ, with

$$θ = \frac{π}{2}$$

being at the north pole) of the electrode locations in the spherical coordinate. Therefore, to estimate the optical flows of the connected components (the contiguous non-zero pixels in I_{BW} in spherical coordinates, based on the calculated optical flows in the 2D images, the following mappings were defined for the vertical and horizontal optical flow magnitudes:

$${V}_{x} ( {ϕ}_{x} , {θ}_{y} ) = r Δ ϕ {V}_{x}^{2 D} ( x , y ) cos ( {θ}_{y} ) \qquad ( 1 )$$

$${V}_{y} ( {ϕ}_{x} , {θ}_{y} ) = r Δ θ {V}_{y}^{2 D} ( x , y )$$

where:
r is an average human head radius (i.e., r=75 mm in various embodiments);
Vₓ^{2D} and V_{y}^{2D} are the horizontal and vertical magnitudes of the estimated optical flow at (x, y) on the 2D plane;
Vₓ(ϕₓ, θ_{y}) and V_{y}(ϕₓ, θ_{y}) are their corresponding magnitudes on the spherical model of the scalp;
(θₓ, θ_{y}) is the corresponding spherical coordinate of the (x, y) location in the 2D plane; and
Δϕ and Δθ are the azimuthal and polar resolution of each pixel in the 2D images.

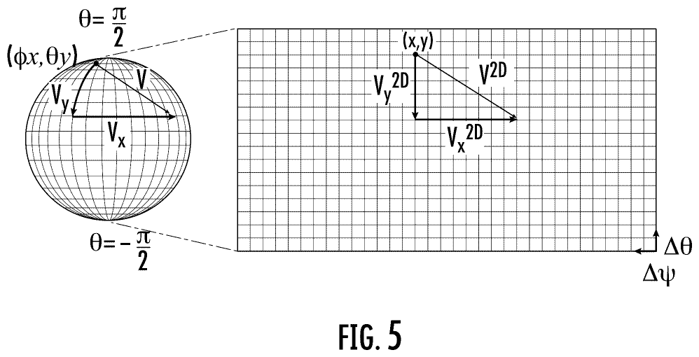

FIG. 5 shows an example of the mapping of an optical flow on the scalp spherical surface. The mapped V=(Vₓ, V_{y}) spherical optical flows were used instead of the original 2D optical flows V^{2D}=(Vₓ^{2D}, V_{y}^{2D}) throughout the next steps of the method. It is worth mentioning that V=(Vₓ, V_{y}) is an estimation of the extent of movements for the connected components in mm on the spherical scalp surface rather than in pixels on the 2D images.

This makes it easier to impose constraints on the speed of propagation of connected components for the detection of SD wavefronts.

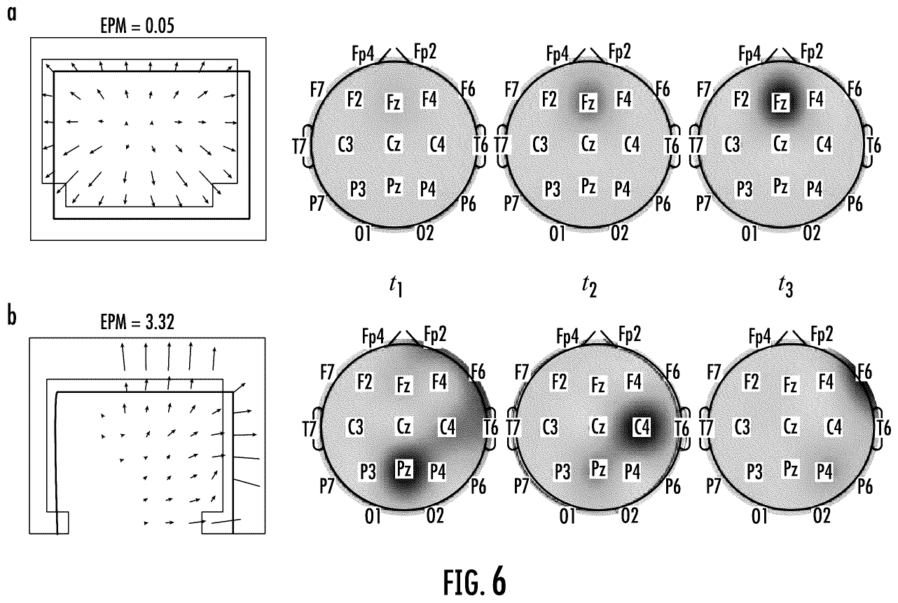

Quantization of Orientations—At step 316, bounding boxes (i.e., BBox) are assigned to the connected components in I_{BW}, shown as 315 in FIG. 3. An orientation histogram for each BBox is calculated and the orientations of optical flows are quantized to finally extract prominent direction(s) of propagation in each BBox. There is an additional constraint on the effective propagation of each BBox before quantization. This additional step was designed to reject the pop-up/fade types of transition of the connected components in the binary images around each electrode location in I_{BW}. FIG. 6a shows an example of pop-up/fade transition in the binary images, while FIG. 6b shows a BBox with significant effective propagation. The low density of the EEG grid makes it impossible to capture small movements of SD wavefronts unless they propagate sufficiently across the 2D planes. Therefore, to reduce the false alarms because of these popup/fade transitions, and only considering the propagating depressions across scalp electrodes, non-propagating Boxes are detected and removed. The effective propagation measure or EPM for each BBox is defined and calculated, based on the estimated optical flows, as follows:

$$EPM = \sqrt{{( \frac{1}{{N}_{o p t}} {\sum }_{i = 1}^{{N}_{o p t}} ‖ {V}_{i} ‖ cos ( {α}_{i} ) )}^{2} + {( \frac{1}{{N}_{o p t}} {\sum }_{i = 1}^{{N}_{o p t}} ‖ {V}_{i} ‖ sin ( {α}_{i} ) )}^{2}} \qquad ( 2 )$$

where:
∥Vᵢ∥ and αᵢ are the magnitude and orientation of the i^{th} optical flow in the Bbox; and
Nₒₚₜ is the total number of optical flows in that BBox.

The first term under the square root in Eq. (2) captures the average horizontal magnitude, and the second term captures the average vertical magnitude of the flows in a given BBox. EPM can take values between 0 and 1, where EPM=0 indicates that all optical flows in the BBox are directed either inward (a fading connected component), outward (a popping-up connected component), or have zero magnitudes (no movement in the corresponding connected component). A threshold on EPM values of BBoxes is applied and the BBoxes and their optical flows are removed if EPM<Thr₂, where Thr₂ is a learnable parameter in the method.

The remaining optical flows are quantized using the orientation histograms. Due to the low-resolution EEG grid, a coarser orientation histogram is used with, in one embodiment, 8 bins of 45° each.

Orientations Bounding Boxes (OBBox)—The orientation bounding boxes (OBBox, 317 in FIG. 3) are extracted using the quantized orientations of optical flows.

Scoring OBBoxes—At step 318, OBBoxes are scored based on the consistency of propagation. A spatial radius of 7 cm (to cover the large inter-electrode distances in this low-resolution EEG grid) is used and a temporal range of Thr₃ min (Thr₃ min before and Thr₃ min after the current frame) to find the neighbors of each OBBox. The algorithm learns the temporal range of Thr₃ through the training process, described below. Speed constraints were imposed on the propagating wavefronts and the OBBoxes with very fast (>8 mm/min) or very slow (<0.5 mm/min) propagation were removed. The number of matching OBBoxes for each of the remaining boxes was counted, and the count is considered as a spatiotemporal score to each OBBox. In addition, the temporal score for each OBBox to measure the consistency in the propagation of wavefronts over time is calculated. If the fraction of the number of frames with non-zero temporal scores over the total number of frames in the Thr₃ min neighborhood is less than Thr₄ (i.e., only a small number of frames contribute to the spatiotemporal score), the corresponding OBBox is removed (i.e., it is assigned a zero spatiotemporal score). Thr₄ is another learnable parameter in the method and takes values between 0 (no contributing frame to the score of the OBBox) and 1 (all frames in the temporal neighborhood of the current frame, defined by Thr₃, contribute to the score of the corresponding OBBox).

Stitching Process and the Final Decision on Detection—At step 320, OBBoxes with small spatiotemporal scores (less than 1% of the maximum available score at each frame) are rejected and the frames with small frame scores (scores of less than 5% of the median of the frame scores) are also rejected. As the final step, the selected frames are stitched together using a sliding time window (2 min in one embodiment) to obtain the final temporal detection output Tₒᵤₜ (1=detected SD at the corresponding frame, 0=no SD wavefront was detected at the corresponding frame).

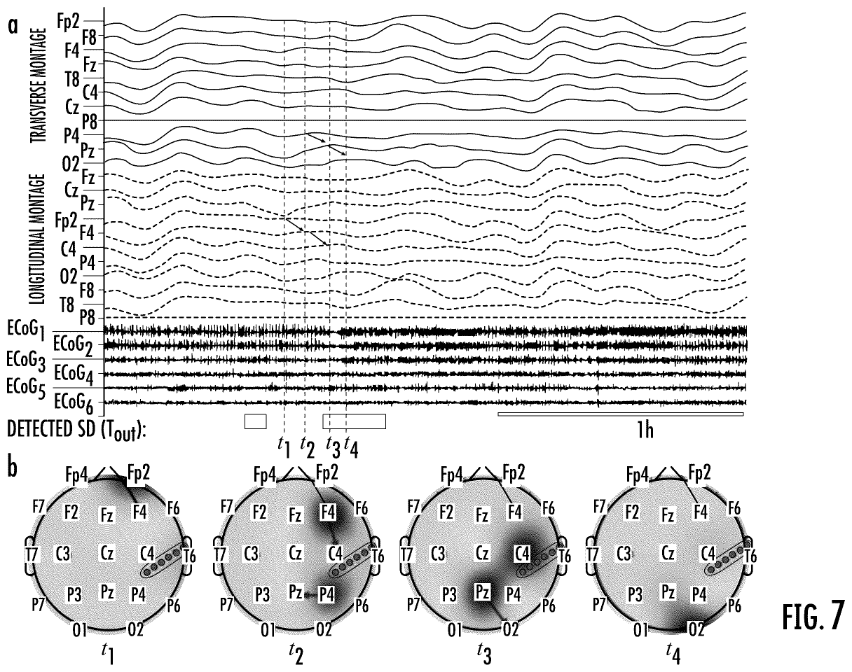

FIG. 7 shows a visualization of SD detection, with a single isolated SD event in a ^{˜}3-hour time window of a single isolated spreading depolarization (SD) event with right decompressive hemicraniectomy (DHC). FIG. 7a are time traces of S_{xcorr} and electrocorticography (ECoG) signals, where four time-points of the selected SD event are marked as t₁, t₂, t₃, t₄ with maximum depressions at Fp2, (F4, P4), (C4, Pz), and O2 respectively. FIG. 7b shows the scalp topography of SD depressions at the four corresponding time points. The intracranial ECoG strip is located around the right temporoparietal lobe. The detected events (Tₒᵤₜ=1) using the disclosed method are marked with blue strips in FIG. 7a.

Learned Parameters—To generalize the disclosed method and detect and prevent overfitting of the framework, cross-validation was used. The dataset described herein from the 12 patients was divided into sets of train and validation patient groups. The optimal sets of parameters for the disclosed method were determined using the train sets, and the SD detection performance was assessed on the validation set, The performance of the disclosed method was averaged on different validation sets. Specifically, Leave-2-out cross-validation (L2O CV) was used. Two patients out of the total 12 patients were chosen and their data was left out for validation in

$$( \begin{matrix}12 \\ 2\end{matrix} ) = 66$$

different ways. For each of these 66 choices, the parameters of the disclosed method were finetuned using the 10-patient training set as described below.

Using the defined performance metrics in the previous section, the parameters (Thr₁^{opt}, Thr₂^{opt}, Thr₃^{opt}, Thr₄^{opt}) were optimized for the best possible train performance (i.e., lowest possible false positive rate (FPR) while maintaining a good detection power, high true positive rate (TPR) and precision as measured by a high positive predictive value (PPV)). This optimized set of parameters was then used to evaluate the performance of the method on the corresponding validation set. A brute-force grid search was used to find the best set of parameters. The following are the training and validation steps for each pair of train-test sets: (i) using the values in the search grids and for each set of (Thr₁, Thr₂, Thr₃, Thr₄), the performance of the method was evaluated on the train set; (ii) a TPR threshold (Thr_{TPR}) was then applied on the train performance, and among the sets of parameters with train TPR>Thr_{TPR}, the one with the minimum FPR was chosen as the optimized set; (iii) the method, using the optimized set of parameters, was applied on the validation set to obtain the validation performance. These steps were repeated for all of the train-validation pairs and the train and validation performance was averaged across all of the 66 pairs of train-validation sets. The average performance for different Thr_{TPR} values in the range of [0.5, 0.85] was calculated; and (iv) the optimal operating point (i.e., Thr_{TPR}^{opt}) and its corresponding optimal set of parameters were found using the underfitting-overfitting (also known as biasvariance) tradeoff. A cross-validation root-mean-square error (RMSE) was defined using the averaged TPR and PPV values as:

$${ϵ}_{CV} = \sqrt{{( 1 - TPR )}^{2} + {( 1 - P P V )}^{2}} \qquad ( 3 )$$

The average validation performance corresponding to the minimum cross-validation error ϵ_{CV} with PPV≥0.5 was reported as the generalization performance. PPV=0.5 is the threshold at which only half of the detected intervals are true positives and the other half are false positives.

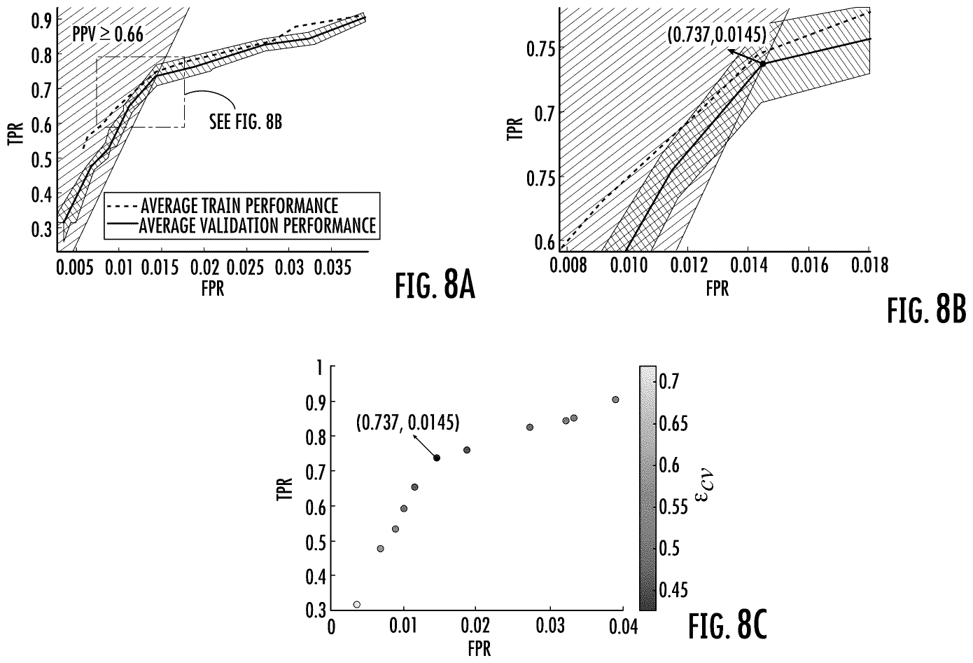

The TPR-FPR tradeoff was captured in a receiver operating characteristic (ROC) curve. The threshold averaging method was used to generate the average train and validation curves. FIG. 8a shows the average train (solid blue line) and validation (solid red line) ROC curves of the performance of the method in the detection of SDs using scalp EEG Delta band ([0.5, 4] Hz) with TPR values along the vertical axis and FPR values along the horizontal axis. The PPV line of PPV=0.66 is overlaid on top of the ROC curves, where the PPV>0.66 region is located above the corresponding PPV line. FIG. 8b shows the cross-validation error (ϵ_{CV}), color-coded across different points in the validation ROC curve, where the point with the minimum error (i.e., optimal operating point) is marked. Based on the results, using Delta frequency band scalp EEG, the method achieves an average validation performance of TPR=0.74±0.03 (12,303 of 16,685 total SD windows were detected) in the PPV>0.66 ROC region, with FPR<0.015 (0.0145±7.57×10−4, 6339 of 437,849 total non-SD windows were falsely detected). All the reported results are within a 95% confidence interval. This operating point in the average ROC curve corresponds to an optimal set of parameters as Thr₁^{opt}−0.3, Thr₂^{opt}=0.6, Thr₃^{opt}=0.69 and Thr₄^{opt}=2.

These parameters have the smallest cross-validation error of 0.4256, while the parameters with higher TPR show an increasing trend in the cross-validation error (i.e., overfitting), and points with lower TPR have larger error as well (i.e., underfitting). Overfitting is inevitable due to the small number of patients in the initial dataset. It is expected that the method will achieve a better validation performance using a larger dataset for the training process.

Estimation of Speed of Propagation—The speed of propagation of detected SD events using the optimized performance parameters in the Delta band can be estimated. For each true detection event (connected 1's in Tₒᵤₜ which laid within At temporal distance of the annotated SD events), averaged over the magnitude of optical flows of the corresponding detected OBBox in the scalp spherical coordinates ∥V∥=\sqrt{Vₓ²+V_{y}²}.

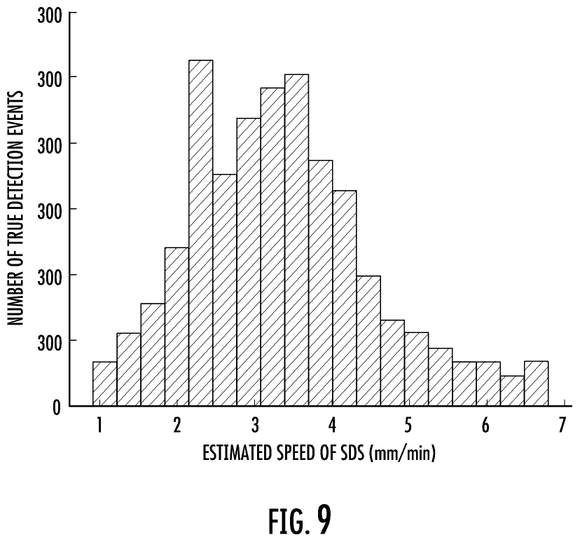

FIG. 9 shows a histogram of the estimated speed of propagation for the true detection events in the Delta band. Although no ground truth is available for the speed of propagation of annotated SD events in the dataset, this helps in understanding and comparing the range of SD speeds in the scalp EEG with the available scientific literature. Based on the results, the estimated speed ranges from 0.9 to 6.8 mm/min, which is a slightly smaller range in comparison to the imposed speed constraints in the method ([0.5, 8] mm/min), with the largest population around 3.6 mm/min. This observation is consistent with the widely reported range of 1-8 mm/min in the literature.

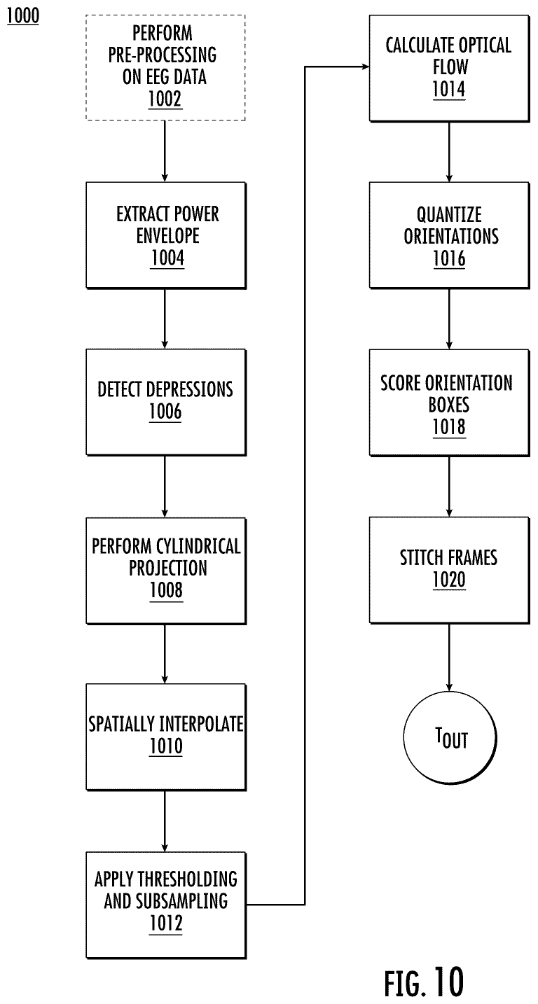

FIG. 10 is a flowchart showing steps of the disclosed method 1000. At 1002, recorded or live EEG data is optionally preprocessed by application of a bandpass filter and downsampling, as previously described. In addition, poor quality segments of the EEG signal may be masked, and outliers may be removed from the data. At step 1004, the power envelope is extracted from the preprocessed EEG data. The signal at each electrode is first normalized and epochs are extracted using overlapping time widows. AT step 1006, depressions are detected in the power envelope. A first-order derivative kernel is used to extract the EEG power depressions. At step 1008, a cylindrical projection is performed to project the power depressions to corresponding scalp locations in a 2D plane. At steps 1010-1012, the image resulting from the cylindrical projection is spatially interpolated, thresholded and subsampled to obtain a binary image where the depression wavefronts were captured as white contiguous pixels. At step 1014, an optical flow is calculated based on the binary image to estimate movement of the wavefronts. At step 1016, dominant directions of propagation of the wavefronts are determined using quantization of orientations. At step 1018, a score is determined based on the consistency and speed of the propagation of the wavefront as determined by the quantization step. At step 1020, candidate frames are stitched together to show an overall movement of the wavefront and a determination is made of whether or not the wavefront represents a SD.

As would be realized by one of skill in the art, the method can be practiced using a system comprising a standard EEG machine and a computing system comprising a processor, a non-transitory storage device and software, stored in the non-transitory storage device that, when executed by the processor, implements the steps of the framework. The EEG data may be analyzed in real-time or may be recorded for later analysis.

As would further be realized by one of skill in the art, many variations on implementations discussed herein which fall within the scope of the invention are possible. Moreover, it is to be understood that the features of the various embodiments described herein were not mutually exclusive and can exist in various combinations and permutations, even if such combinations or permutations were not made express herein, without departing from the spirit and scope of the invention. Accordingly, the method and apparatus disclosed herein are not to be taken as limitations on the invention but as an illustration thereof. The scope of the invention is defined by the claims which follow.

1. A method comprising:
receiving EEG data;
determining a power envelope of the EEG data for each EEG electrode;
detecting depressions in the power envelope of each EEG electrode;
projecting the detected depressions from each EEG electrode on a 2D plane;
obtaining a binary image from the projection of the detected depressions;
estimating movement of wavefronts in a time series of binary images;
determining a dominant direction of propagation of the wavefronts;
scoring each wavefront based on a consistency of speed and propagation; and
selecting candidate frames based on the score for each wavefront; and
stitching together selected frames using a sliding time window to obtain a final temporal detection indicating presence of a spreading depolarization (SD) wavefront.

2. The method of claim 1 wherein detecting depressions in the power envelope for each EEG electrode comprises:
detecting falling edges of the power envelope.

3. The method of claim 2 further comprising:
cross-correlating the power envelope for each EEG electrode with a first-derivative kernel such that the falling edges of the power envelope appear as peaks in a cross-correlation curve.

4. The method of claim further comprising:
rectifying the cross-correlation curve for each EEG electrode to isolate the peaks.

5. The method of claim 1 wherein the detected depressions are projected on a 2D plane using cylindrical projection of locations of the EEG electrodes.

6. The method of claim 1 further comprising:
performing spatial interpolation on the projected depressions to produce a smooth 2D image;
thresholding the smooth 2D image to obtain the binary image.

7. The method of claim 6 wherein the thresholding comprises:
setting a pixel value to 0 in the smooth 2D image when the value of the pixel is below a first threshold; and
setting the pixel value to 1 in the smooth 2D image when the pixel value is above the first threshold to create the binary image.

8. The method of claim 7 wherein pixels representing a location of each EEG electrode are present in the binary image.

9. The method of claim 8 wherein estimating movement of SD wavefronts in the time series of binary images comprises:
spatially subsampling each binary image in the time series to reduce inter-electrode distances in each binary image; and
applying an optical flow calculation to the time series of binary images to determine a magnitude of speed and direction of propagation of the SD wavefront.

10. The method of claim 9 wherein determining a dominant direction of movement of the wavefronts comprises:
assigning bounding boxes to connected components in the time series of binary images;
calculating an orientation histogram for each bounding box;
quantizing the orientation of the optical flows based on the quantization; and
extracting a dominant direction of propagation for each bounding box based on the orientations.

11. The method of claim 10 further comprising:
removing non-propagating bounding boxes.

12. The method of claim 11 further comprising:
calculating an effective propagation measure for each bounding box; and
removing bounding boxes having an effective propagation measure below a second threshold.

13. The method of claim 12 further comprising:
removing all bounding boxes having a magnitude of speed outside of a predetermined range.

14. The method of claim 13 further comprising:
finding spatial and temporal neighbor frames for each remaining bounding box;
wherein a frame is a temporal neighbor of another frame in the bounding box if its temporal distance is within a temporal range is given by a third threshold.

15. The method of claim 14 further comprising:
determining a spatiotemporal score for each remaining bounding box based on the number of matching bounding boxes.

16. The method of claim 15 further comprising:
determining a temporal score for each remaining bounding box;
wherein the temporal score is based on a ratio of the remaining frames of the bounding box having a non-zero temporal score to the total number of frames of the bounding box.

17. The method of claim 16 further comprising:
removing the bounding box if the total number of frames having a temporal neighbor is less than a fourth threshold.

18. The method of claim 17 further comprising:
stitching together remaining frames using a sliding time window to obtain a final determination of the existence of an SD wavefront.

19. The method of claim 18 wherein the first, second, third and fourth thresholds are learned parameters.

20. The method of claim 19 wherein the learned parameters are selected based on performance on a validation dataset after being optimized on a training dataset.
